(19) United States
(12) Patent Application Publication
(10) Pub. No.: US 2013/0191933 A1
(43) Pub. Date: Jul. 25, 2013

(54) METHODS OF TREATING PAIN AND MORPHINE TOLERANCE VIA MODULATION OF HEDGEHOG SIGNALLING PATHWAY

(71) Applicants: Michael J. Galko, Houston, TX (US); Daniel T. Babcock, Madison, WI (US); Howard B. Gutstein, Bellaire, TX (US)

(72) Inventors: Michael J. Galko, Houston, TX (US); Daniel T. Babcock, Madison, WI (US); Howard B. Gutstein, Bellaire, TX (US)

(73) Assignee: BOARD OF REGENTS, THE UNIVERSITY OF TEXAS SYSTEM, Austin, TX (US)

(21) Appl. No.: 13/703,587

(22) PCT Filed: Jun. 7, 2011
(86) PCT No.: PCT/US11/39387

§ 371 Date: Apr. 8, 2013

Related U.S. Application Data
(60) Provisional application No. 61/353,716, filed on Jun. 11, 2010.

(51) Int. Cl. A61K 31/4355; C07K 14/435; A61K 31/485

(52) U.S. Cl. CPC A61K 31/4355; A61K 31/485; C07K 14/43581
USPC 800/9; 514/278; 514/282

(57) ABSTRACT

Described herein are methods of treating nociception by administering to a subject in need thereof a therapeutic amount of one or more compounds which modulate the Hedgehog signaling pathway.

### CROSS-REFERENCE TO RELATED APPLICATIONS

This patent application claims priority to U.S. Patent Application Ser. No. 61/353,716 filed Jun. 11, 2010 which is incorporated by reference herein in its entirety

### FIELD OF INVENTION

The present invention is directed to novel methods of treating nociception, allodynia and hyperalgesia by using compounds which modulate the Hedgehog Signaling Pathway.

### STATEMENT REGARDING FEDERALLY FUNDED RESEARCH OR DEVELOPMENT

None.

### THE NAMES OF PARTIES TO A JOINT RESEARCH AGREEMENT

None.

### REFERENCE TO SEQUENCE LISTING

This disclosure includes a sequence listing submitted as a text file pursuant to 37 C.F.R. §1.52(e)(v) named sequence listing.txt, created on May 17, 2010 with a size of 9,568 bytes, which is incorporated herein by reference. The attached sequence descriptions and Sequence Listing comply with the rules governing nucleotide and/or amino acid. sequence disclosures in patent applications as set forth in 37 C.F.R. §§1.821-1,825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984). The symbols and format used for nucleotide and amino acid sequence data comply with the rules set forth in 37 C.F.R. §1.822,

### BACKGROUND OF THE INVENTION

The current anti-nociceptive drugs include opioids such as morphine which are effective at treating many types of pain. Opioids, however, are completely ineffective against other types of severe pain, and lose effectiveness against pain due to the development of analgesic tolerance with repeated dosing. Opioids also have significant adverse side effects such as addiction and respiratory depression. Hence, there has long been a search for other suitable drug targets that might allow one to develop drugs that treat pain hypersensitivity without these adverse side effects.

### BRIEF SUMMARY OF INVENTION

Novel methods of treating nociception by administering one or more compounds that modulate the Hedgehog signaling pathway and/or a component thereof are provided herein. Such methods include administering a therapeutic amount of compound that is an inhibitor of the binding of the Hedgehog ligand to the Patched protein, an agonist of the Patched protein or an inhibitor of the Smoothened or Gli proteins. Methods of treating allodynia and hyperalgesia are also described. Further provided are Drosophila which express either dominant-negative Patched transgene or constitutively active Smoothened transgene in larval nociceptive sensory neurons and thus exhibit chronic nociceptive sensitization. These latter animals are useful for genetic identification of further gene targets that are downstream of Smoothened in nociceptive sensitization in Drosophila larvae.

### BRIEF DESCRIPTION OF THE DRAWINGS

FIG. 1 is a schematic of the Drosophila Hedgehog signaling pathway. In the canonical Hedgehog signaling pathway (schematized in part above) the Hedgehog receptor, Patched (Ptc), inhibits signal transduction through its coreceptor, Smoothened (Sitio). Binding of Hedgehog (Hh) to Patched relieves this inhibition and allows signal transduction to proceed. The end result of pathway activation is that the transcription factor Cubitus Interuptus, (Ci, a homolog of vertebrate Gli transcription factors) is converted from a repressive form to an active form and subsequently activates transcription of pathway target genes including the transcription factor, Engrailed (En), the BMP-like growth factor, Dpp, and Ptc. In our experiments detailed in the preceding figures, inhibition of positive regulators of the pathway (shown in blue above) or overexpression or activation of negative regulators (shown in red above) leads to loss of development of tissue damage-induced allodynia and hyperalgesia. Thus, the Hedgehog signaling pathway represents a novel and completely unexpected set of potential drug targets for inhibiting the development of pain sensitization.

FIG. 2 is a schematic of the basic Drosophila assay for development of thermal allodynia and hyperalgesia. Adult male Drosophila bearing a nociceptive sensory neuron specific Gal4 driver (ppk1.9-Gal4) were crossed to females bearing a transgene under UAS (Gal4 binding site) control that interferes with Hedgehog signal transduction. Progeny larvae bearing both transgenes or a temperature sensitive mutation in hedgehog were UV-irradiated to induce epidermal tissue damage (see Babcock et al, Current Biology, 2009). Sets of irradiated larvae were then tested for allodynia using a thermal probe set to 38° C. (the highest normally sub-threshold temperature) after 24 hours of recovery (the peak of the allodynia response in control larvae). Alternatively, to test for development of hyperalgesia, the larvae were probed at 45° C. (the lowest noxious temperature at which all control larvae respond with aversive withdrawal) after 8 hours of recovery (the peak of the hyperalgesia response in control larvae). When testing for allodynia, larvae were grouped into three categories: 1. No response (aversive withdrawal) within a 20 s cutoff; 2. slow response between 5 and 20 s of contact with the probe; or, 3. Fast response where aversive withdrawal was observed in less than 5 s. When testing for hyperalgesia, only the time to aversive withdrawal was recorded since all larvae are responsive at 45° C.

FIGS. 3A and 3B show that Drosophila Hedgehog is required for development of thermal allodynia and hyperalgesia. Drosophila larvae bearing either a control mutation (w1118) or a temperature-sensitive allele of hedgehog, hhts2, were reared at the permissive temperature (normal Hedgehog function) of 18° C. In the early third instar larvae were either mock-treated or exposed to 20 mJ/cm2 of UV and reared at either the permissive temperature or the restrictive temperature (hs; no Hedgehog function) of 29° C. A, Allodynia. 24 hours after mock or UV treatment, the larvae were probed with a normally non-noxious 38° C. thermal stimulus to test for development of allodynia. w1118 larvae showed responsiveness after UV that was independent of the rearing temperature, hhts2 larvae, by contrast, exhibited greatly reduced responsiveness at the restrictive temperature (hs) indicating that Hh is required for development of allodynia. B, Hyperalgesia. 8 hours after mock or UV treatment, the larvae were probed with a normally noxious 45° C. thermal stimulus to test for development of hyperalgesia. w1118 larvae showed a faster withdrawal latency that was independent of the rearing temperature while hhts2 larvae showed a reduced withdrawal latency only at the permissive temperature, indicating that Hh signaling is also required for this type of pain sensitization. N=90 larvae per condition.

FIG. 4 shows that Drosophila Hedgehog signaling pathway components are required within larval sensory neurons for development of tissue damage-induced allodynia. Drosophila bearing a nociceptive sensory neuron specific Gal4 driver that allows tissuespecific expression of transgenes under UAS (Gal4 binding site) control were crossed to control flies (w1118) or flies bearing a UAS-transgene targeting a signaling pathway component. IR, inverted repeat; DN, dominant negative, smo=smoothened; ci=cubitus interuptus; en=engrailed; dpp=decapentaplegic; ptc=patched. In the early third instar larval progeny were exposed to 20 mJ/cm2 of UV and reared on standard media for 24 hours before probing with a normally non-noxious 38° C. thermal stimulus to test for development of allodynia. Control larvae (ppk/+) showed a normal distribution of slow (aversive withdrawal between 5 and 20 s) and fast (aversive withdrawal in under 5 s) responders. Larvae bearing transgenes interfering with Hedgehog signal transduction, by contrast, exhibited greatly reduced responsiveness indicating that canonical Hedgehog signaling pathway components, including receptors (ptc, smo), transcription factors (ci, en) and downstream targets (en, dpp) are required for development of allodynia, N=90 larvae per genotype.

FIG. 5 shows that Drosophila Hedgehog signaling pathway components are required within larval sensory neurons for the development of tissue damage-induced hyperaigeisa. Drosophila bearing a nociceptive sensory neuron specific Gal4 driver that allows tissuespecific expression of transgenes under UAS (Gal4 binding site) control were crossed to control flies (w1118) or flies bearing a UAS-transgene targeting a Hh signaling pathway component. IR, inverted repeat; DN, dominant negative. smo=smoothened; ci=cubitus interuptus; en=engrailed; dpp=decapentaplegic; ptc=patched. In the early third instar larval progeny were mock-treated or exposed to 20 mJ/cm2 of UV and reared on standard media for 8 hours before probing with a noxious 45° C. thermal stimulus to test for development of hyperalgesia. Control larvae (ppk/+) showed a normal withdrawal latency of 6.9 s in the absence of UV and 3.8 s after UV exposure. Larvae bearing transgenes interfering with Hedgehog signal transduction did not exhibit a lowered withdrawal latency following UV exposure indicating that canonical Hedgehog signaling pathway components, including receptors (ptc, smo), transcription factors (ci, en) and downstream targets (en, dpp) are required for development of hyperalgesia. N=50 larvae per genotype.

FIG. 6 shows that baseline nociception normal is Hh pathway knockdowns. Drosophila bearing a nociceptive sensory neuron specific Gal4 driver that allows tissuespecific expression of transgenes under UAS (Gal4 binding site) control were crossed to control flies (w/118) or flies bearing a UAS-transgene targeting a Hh signaling pathway component. IR, inverted repeat; DN, dominant negative. smo=smoothened; ci=cubitus interuptus; en=engrailed; dpp=decapentaplegic; ptc patched. Progeny larvae were tested for withdrawal responses at the middle of the response range (45° C., the lowest temperature where all larvae respond with aversive withdrawal) and the upper end of the response range (48° C. the lowest temperature where all larvae respond in under 5 s to contact of the heat probe). In no cases were the recorded withdrawal latencies different from control ppk/+ larvae,

FIGS. 7A and 7B show ectopic Hh causes allodynia and hyperalgesia. In all panels ppk1.9-Gal4 drives (>) expression of the indicated UAS transgenes or no transgene (ppk1.9-Gal4×w¹¹¹⁸=ppk/+) in nociceptive sensory neurons. A. Sensory neuron-specific expression of transgenes that lead to constitutive activation of Hh signaling (UAS-ptc^{DN} or UAS-Smoothened^{□fu}) cause allodyia (responsiveness to the normally sub-threshold temperature of 38° C.) even in the absence of UV irradiation. B. Expression of the same transgenes in A also causes hyperalgesia, exaggerated responsiveness to the supra-threshold temperature of 45° C.

FIG. 8 show that Hh-induced allodynia is independent of TNF signaling. ppk1.9-Gal4 drives (>) expression of the indicated UAS transgenes or no transgene (ppk1.9-Gal4×w¹¹¹⁸=ppk/+) in nociceptive sensory neurons. Activation of TNF signaling through expression of either TNF (Eiger=egr) or activation of Hh signaling through expression of UAS-ptc^{DN} leads to ectopic allodynia even in the absence of tissue damage. When allodynia is activated by TNF, blockade of the TNF receptor, Wengen, dampens this response, wheras knockdown of Smoothened does not. Conversely, when allodynia is activated by activation of Hh signaling, knockdown of Smoothened dampens this response but knockdown of Wengen does not. These results indicate that TNF and Hh signaling act independently and in parallel in mediating allodynia.

FIGS. 9A and 9B show TNF and Hh-induced allodynia both require TRP channel activity. ppk1.9-Gal4 drives (>) expression of the indicated UAS transgenes or no transgene (ppk1.9-Gal4×w¹¹¹⁸ ppk/+) in nociceptive sensory neurons, A. Allodynia caused by ectopic activation of TNF or Hh signaling requires the function of the Painless TRP channel but not the TRPA1 TRP channel. B. Hyperalgesia caused by ectopic activation of Hh signaling requires the TRPA1 TRP channel but not the Painless TRP channel.

FIG. 10 shows that the combination of cyclopamine and morphine reverses pain sensitivity for an extended period in rats. Animals underwent sciatic nerve ligation as described (Chung et al.), and were then allowed to recover for two weeks. Allodynia was measured using mechanical withdrawal threshold (Von Frey hairs). Preoperative thresholds were approx. 14 g, and remained at this level in sham-operated animals. Postoperative baseline thresholds were 3-4 g. Drugs were administered using lumbar puncture under anesthesia. Neither morphine or cyclopamine had analgesic effects when given individually, but the combination completely reversed pain sensitivity for an extended period (MS÷CP-morphine and cyclopamine)

FIG. 11 shows that the combination of cyclopamine and morphine reverses inflammatory hyperalgeisa for an extended period in rats, without the development of morphine tolerance. Animals underwent complete Freund's adjuvant (CFA) injection under anesthesia. Hyperalgesia was then measured using withdrawal latency of the paw to a thermal stimulus (PWL). Preadjuvant latencies were approx. 10-12 s, and remained at this level in sham-operated animals. Post-adjuvant baseline thresholds were 3-4 g. Drugs were administered using lumbar puncture under anesthesia. Morphine alone exhibited analgesic effects, but tolerance developed rapidly. By Day 3, Cyclopamine developed a mild analgesic effect that plateaued. The combination of morphine and cyclopamine completely reversed pain hyperalgeisa for an extended period, without the development of tolerance (MS+CP-morphine and cyclopamine).

FIG. 12 indicates that cyclopamine completely reversed the underlying biochemical processes responsible for the development of morphine tolerance in rats. Drugs were administered daily using lumbar puncture under anesthesia. Tolerance was then assessed using tail flick latency (TFL), or the withdrawal latency of the tail to a thermal stimulus. Baseline (BL) latencies were 3-4 s Animals received morphine alone (MS), morphine and cyclopamine (MS+CP), morphine alone with cyclopamine added on Day 3 (MS+CP3), or morphine alone with cyclopamine added on Day 5 (MS+CP5). On Day 8, all animals received morphine alone. Cyclopamine completely reversed tolerance within two days, even in animals that were completely tolerant. The fact that animals were not tolerant on Day 8 after cyclopamine was removed indicates that it reversed the underlying biochemical processes responsible for the development of morphine tolerance. These findings could have profound implications for our patients that are already extremely tolerant to opioids,

### DETAIL DESCRIPTION OF INVENTION

The secreted morphogen Hedgehog (sometimes referred to herein as “Hit”) regulates several developmental processes in a variety of tissues including embryonic patterning, cell fate specification, axon guidance, and proliferation. However, other than involvement in cellular proliferation and cancer, to date, the role of Hh in the physiology of differentiated tissues is, at best, unclear. Notwithstanding, as described herein, a novel physiological role for Hh signaling in nociception, has been shown, providing new methods of treating pain and morphine tolerance.

Pharmacological blockade of the Hedgehog signaling pathway can be useful in the clinical treatment of pain that often accompanies trauma, surgery or chemotherapy as well as blocking the development of tolerance to opioid analgesics commonly used to treat pain. Furthermore, pharmacological blockade of the pathway can be used to treat neuropathic and inflammatory pain. Since tolerance is a key component underlying addiction, modulation of Hh signaling can also be used as a therapeutic target for the treatment of addiction. Described below are the genetic experiments in Drosophila that led to the discovery of a role for Hh signaling in nociception. The vertebrate experiments are provided next, showing this role is evolutionarily conserved.

Overview of Hh Signaling. The Hh signaling pathway is critical in the formation of many organs and for axon guidance in the nervous system. Although it is well-studied in developmental contexts, this pathway has never been implicated in pain signaling in any system.

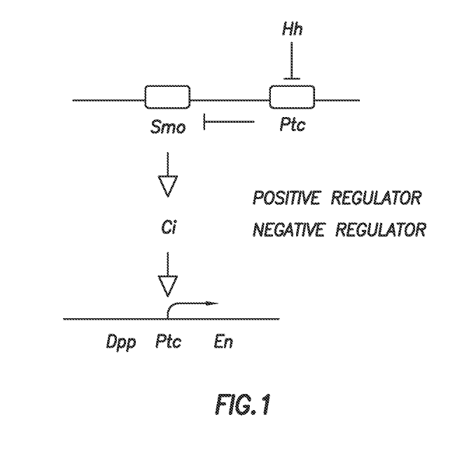

In the canonical Hedgehog signaling pathway, schematized in part in FIG. 1, the Hedgehog receptor, Patched inhibits signal transduction through its co-receptor, Smoothened. Binding of Hedgehog (Hh) to Patched relieves this inhibition and allows signal transduction to proceed. The end result of pathway activation is that the transcription actor Cubitus Interuptus, (“Ci,” a homolog of vertebrate Gli transcription factors) is converted from a repressive form to an active form and, together with its cofactor, subsequently activates transcription of pathway target genes including the transcription factor, Engrailed (En), the BMP-like growth factor, Dpp, and the Hh-binding protein, Ptc. Conservation of the pathway components between Drosophila and vertebrates is shown in Table 1 below.

“In Drosophila, Hedgehog signaling is initiated by the binding of Hedgehog ligand to Patched (”Ptc“) which is a 12-transmembrance protein receptor. Ptc acts as an inhibitor of Smoothened (”Smo“), a 7 transmembrane protein related to the Frizzled family of Wnt receptors and to other 7 transmembrane G protein-coupled receptors. Downstream of Smo is a multi-protein complex known as the Hedgehog signaling complex (“HSC”) which comprises the transcription factor Cubitus interruptus (“Ci”), the serine/threonine kinase Fused (“Fu”), the kinesin-like molecule Costal 2 (Cos2) and Supressor of fused (Sufu). Cos2 also binds to protein kinase A (pkA), protein kinase CK1 (formerly casein kinase 1) and glycogen synthase kinase 3 (GSK2), which are other kinases implicated in the Hedgehog signaling pathway.

In the absence of ligand, Ptc represses Smo, preventing the activation of Hedgehog signaling via proteolytic cleavage of Ci. Cleavage results in a repressor form of Ci, which enters the nucleus and inhibits Hedgehog target gene expression. In the presence of Hedgehog (Hh), the inhibitory effects of Ptc on Smo are relieved. Smo becomes phosphorylated by PKA and CK1. Then PKA, CK1 and GSK3 are released from Cos2, precluding the generation of the repressor form of Ci. Full length Ci is no longer inhibited by Sufu and is therefore free to enter the nucleus to induce the transcription of Hedgehog target genes such as engrailed, Ptc and decapentaplegic.”

Furthermore, “Hedgehog is synthesized as a precursor, which undergoes an autoproteolytic cleavage to liberate a 19 kDa N-terminal fragment (N-Hh), which displays all known signaling properties and a slightly larger C-terminal peptide fragment that has no apparent function other than to catalyse cleavage. The auto-processing reaction provides a trigger for the addition of a cholesterol moiety to the C-terminal of N-Hh. The N terminus is modified via the addition of a palmitate molecule by the acyltransferase. Skinny Hedghog (Skn)—also known as Central missing (Cmn), Rasp and Sightless. Membrane tethered Hedgehog proteins initiate signaling in the nearby vicinity of the producing cell or Hedgehog proteins form multimeric complexes in which the hydrophobic moieties cluster together in an inner core allowing diffusion of ligand and long range signaling. Dispatched (Disp) and Tout-velu (Ttv) mediate Hedgehog release and diffusion. Disp is required for release of membrane anchored Hedgehog protein and Ttv regulates the synthesis of proteoglycans enabling the movement of Hedgehog ligands thereby facilitating long range signaling.” Hooper, C., Department of Neuroscience, Institute of Psychiatry, Kings College London, Denmark Hill, London, SE5 8AF, A Mini-review of the Current Understanding of the Hedgehog Signaling Pathway, Abcam.com, text as quoted is incorporated herein by reference.

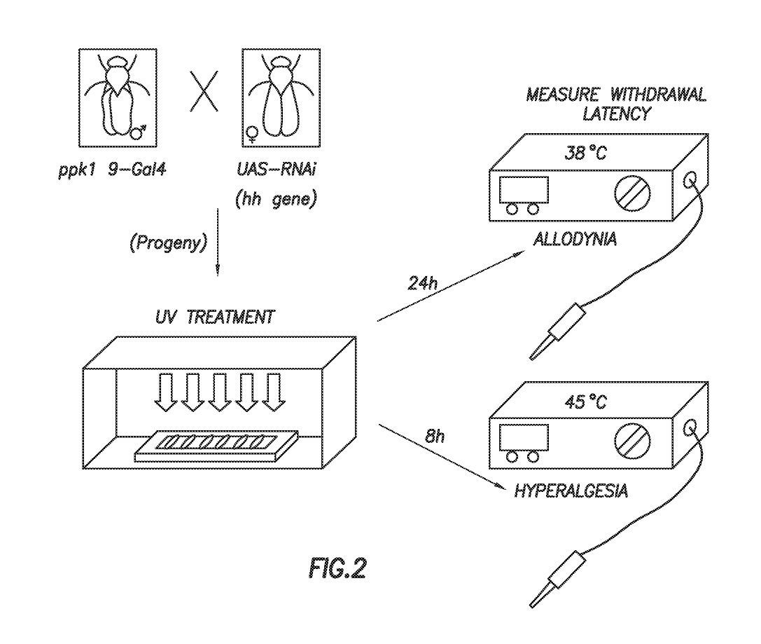

Drosophila Model of Tissue Damage-Induced Nociceptive Hypersensitivity. A Drosophila model of tissue damage-induced nociceptive hypersensitivity was developed (FIG. 2). Using this model, larvae were used to show both a lowering of the nociceptive threshold (allodynia) and an exaggerated responsiveness to noxious stimuli (hyperalgesia) Babcock, D. T., et al., Cytokine Signaling Mediates UV-Induced Nociceptive Sensitization in Drosophila Larvae, Curr Biol 19, 799-806 (2009). With this model, we demonstrated that inhibiting canonical Hh signaling components in nociceptive sensory neurons prevents both thermal allodynia and hyperalgesia following ultraviolet (UV) damage, without interfering with baseline nociception. The assay involves UV-irradiating Drosophila larvae to create a stripe of apoptotic cell damage to the dorsal barrier epidermis (skin). Irradiated larvae are then probed with a custom-designed thermal heat probe to assess their thermal pain responsiveness. When testing for allodynia, larvae were grouped into three categories: 1. No response (aversive withdrawal) within a 20 s cutoff; 2. Aversive withdraw” between 5 and 20 seconds of contact with the probe (slow response); or, 3, aversive withdrawal was observed in less than 5 seconds (fast response). When testing for hyperalgesia, only the time to aversive withdrawal (withdrawal latency) was recorded since all larvae are responsive at 45° C.

Nociceptive Neurons Protect Drosphilia Larvae from Parasitoid Wasps. As in vertebrates, larvae with damaged tissues exhibit both allodynia (responsiveness to previously sub-threshold stimuli) and hyperalgesia (exaggerated responsiveness to noxious supra-threshold stimuli). Once we established the basic assay, we uncovered which genes are required within nociceptive sensory neurons for development of thermal allodynia by using an in vivo RNAi strategy to knock down genes solely in the neurons previously shown to be required for the aversive withdrawal behavior that accompanies receipt of a noxious thermal stimulus. This screen specifically targeted genes encoding ion channels and (i-Protein-coupled receptors (GPCRs). As a result, we found that knockdown of the Hedgehog (Hh) signaling co-receptor, Smoothened, which is encoded by the Smoothened gene, blocks development of allodynia.

Drosophila Hedgehog mutants fail to develop tissue damage-induced allodynia and hyperalgesia: Cells within damaged tissues release a number of factors that can act on neighboring cells. Some of these are secreted factors such as lipids, cytokines, neurotransmitters, and ions that play a large role in nociceptive hypersensitivity. Others are morphogens, including Wingless, Decapentaplegic (Dpp), and Hh, which in the context of damaged tissues can induce compensatory proliferation. Fan, Y., et al, Distinct Mechanisms of Apoptosis-Induced Compensatory Proliferation in Proliferating and Differentiating Tissues In The Drosophila Eye, Dev Cell, 14, 399-410 (2008); Huh, J. R., et al, Compensatory Proliferation Induced by Cell Death in the Drosophila Wing Disc Requires Activity of the Apical Cell Death Caspase Drone in a Nonapoptotic Role, Curr Biol 14, 1262-1266 (2004); Perez-Garijo, A., et al., Caspase Inhibition During Apoptosis Causes Abnormal Signalling and Developmental Aberrations in Drosophila, Development 131, 5591-5598 (2004); Perez-Garijo, A., et al., The Role of Dpp and Wg in Compensatory Proliferation and in the Formation of Hyperplastic Overgrowths Caused by Apoptotic Cells in the Drosophila Wing Disc, Development 136, 1169-1177 (2009); Ryoo, H. D., et al, Apoptotic Cells can Induce Compensatory Cell Proliferation through the JNK and the Wingless Signaling Pathways, Dev Cell 7, 491-501 (2004). Whether the morphogens produced within damaged tissues can alter the sensitivity of nociceptive sensory neurons has been unknown to date. But as described herein, we established the role of Hh in nociceptive hypersensitivity.

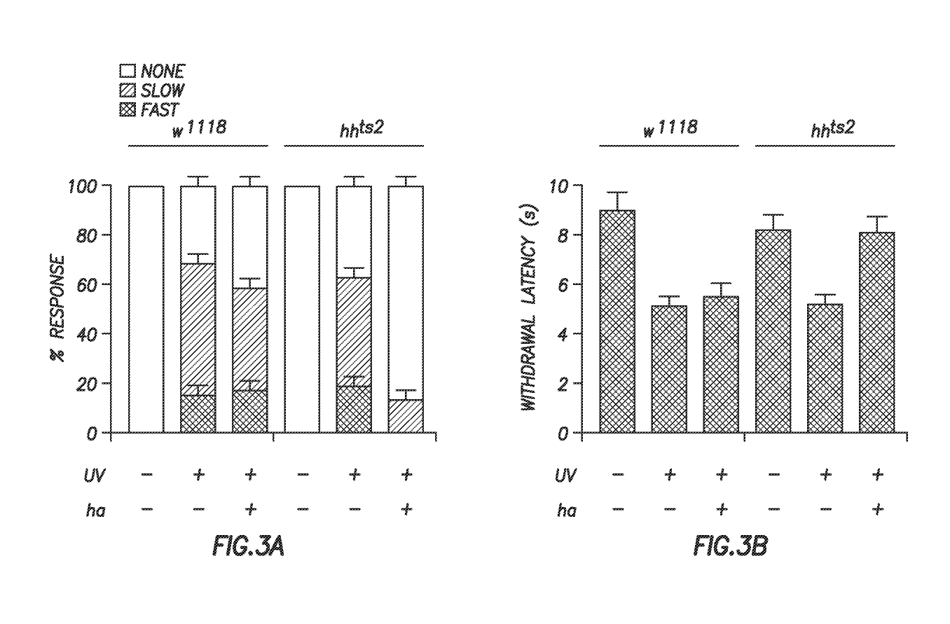

We first tested whether larvae bearing a temperature-sensitive allele of hh (hh^{ts2}) developed thermal allodynia and hyperalgesia following UV damage. Ma, C., et al., The Segment Polarity Gene Hedgehog is Required far Progression of the Morphogenetic Furrow in the Developing Drosophila Dye, Cell 75, 927-938 (1993). Drosophila larvae bearing either a control mutation (w¹¹¹⁸) or a temperature-sensitive allele of hedgehog, hh^{ts2} were reared at the permissive temperature (normal Hedgehog function) of 18° C. In the early third instar, larvae were either mock-treated exposed to 20 mJ/cm² of UV and reared at either the permissive temperature or the restrictive temperature (heat schock, hs; no Hedgehog function) of 29° C. FIG. 3A shows the data related to allodynia. 24 hours after mock or UV treatment, the larvae were probed with a normally non-noxious 38° C. thermal stimulus to test for development of allodynia. w¹¹¹⁸ larvae showed responsiveness after UV that was independent of the rearing temperature. hh^{ts2} larvae, by contrast, exhibited greatly reduced responsiveness at the restrictive temperature (hs) indicating that Hh is required for development of allodynia. FIG. 3B shows the data related to hyperalgesia., 8 hours after mock or UV treatment, the larvae were probed with a normally noxious 45° C. thermal stimulus to test for development of hyperalgesia w¹¹¹⁸ larvae showed a faster withdrawal latency that was independent of the rearing temperature while hh^{ts2} larvae showed a reduced withdrawal latency only at the permissive temperature, indicating that Hh signaling is also required for this type of pain sensitization. N=90 larvae per condition. Taken together, these results suggest that Hh signaling is required for both thermal allodynia and hyperalgesia after UV damage and distinguish this pathway from TNF signaling, which is only required for development of thermal allodynia.

Hh signaling acts within larval nociceptive sensory neurons to mediate nociceptive sensitization. To examine whether Hh signaling acts within nociceptive sensory neurons and through the canonical pathway, we expressed various transgenes that target components of the Hh signaling pathway using a Gal4 driver (ppk1.9-Gal4) specific to these neurons. Ainsley, J. A. et al., Enhanced Locomotion Caused By Loss of the Drosophila DEG/ENαC Protein Pickpocket1, Curr. Biol. 13, 1557-11563 (2003). As noted above, in canonical Hh signaling, Hh binds to and inhibits its receptor, Patched (“Ptc”), Hooper, J. E. & Scott, M. P., Communicating with Hedgehogs, Nat. Rev. Mol. Cell Biol. 6, 306-317 (2005). This binding relieves inhibition of the transmembrane protein, Smoothened (”Smo“), and leads to signal transduction through the kinase Fused, the end result of which is activation of the transcription factor Cubitus Interuptus (“Ci”), Activated Ci then turns on expression of target genes such as decapentaplegic (“dpp”) and engrailed (“en”).

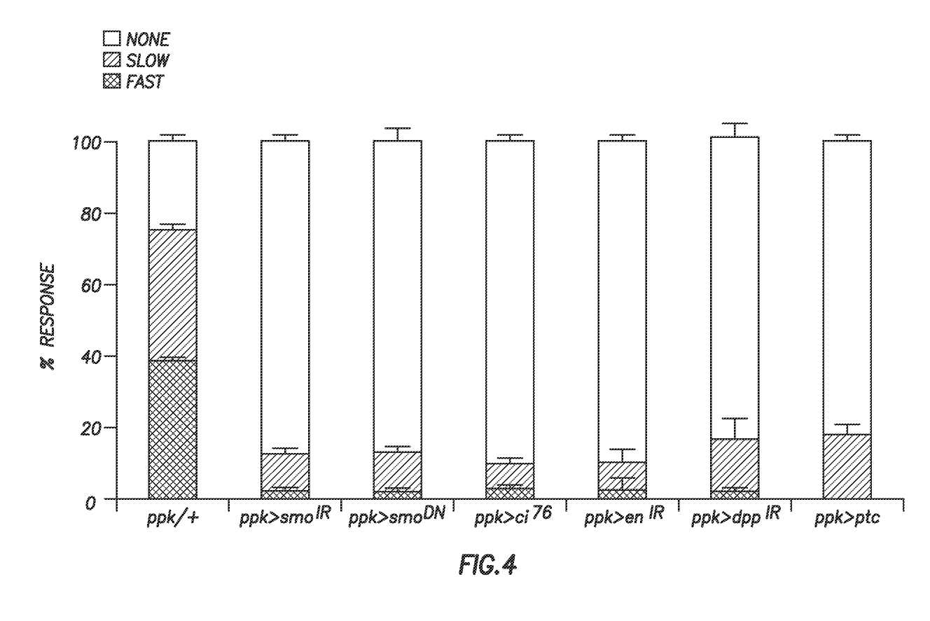

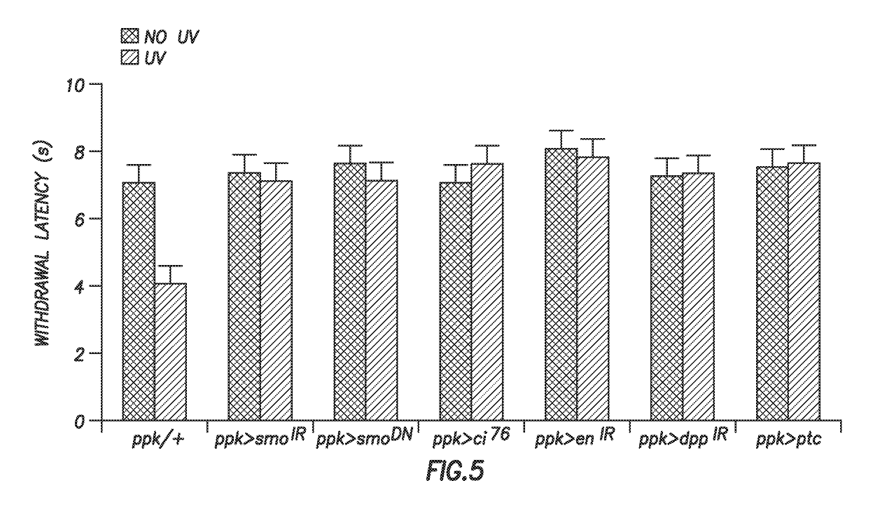

As shown in FIGS. 4 and 5, to determine whether Hedgehog signaling pathway components are required within sensory neurons for development of tissue damage-induced allodynia, Drosophila bearing a nociceptive sensory neuron specific Gal4 driver were crossed to control flies (w¹¹¹⁸) or flies bearing a UAS-transgene targeting a Hh signaling pathway component I R, inverted repeat; DN, dominant negative. The Gal4 driver allows tissue specific expression of transgenes under UAS (Gal4 binding site) control. In FIGS. 4 and 5 and as otherwise sometimes referred to herein, “smo” denotes Smoothened; “ci” denotes cubitus interuptus; “en” denotes engrailed; “dpp” denotes decapentaplegic; “ptc” denotes patched. In the early third instar, larval progeny were exposed to 20 mJ/cm² of UV and allowed to recover on standard media for 24 hours before probing with a normally non-noxious 38° C. thermal stimulus to test for development of allodynia, Control larvae (ppk/+) showed a normal distribution of slow (aversive withdrawal between 5 and 20 seconds) and fast (aversive withdrawal in under 5 seconds) responders. Larvae bearing trans-genes interfering with Hedgehog signal transduction, by contrast, exhibited greatly reduced responsiveness indicating that canonical Hedgehog signaling pathway components, including receptors (ptc, smo), transcription factors (ci, en) and downstream targets (en, dpp) are required for development of allodynia, N=90 larvae per genotype.

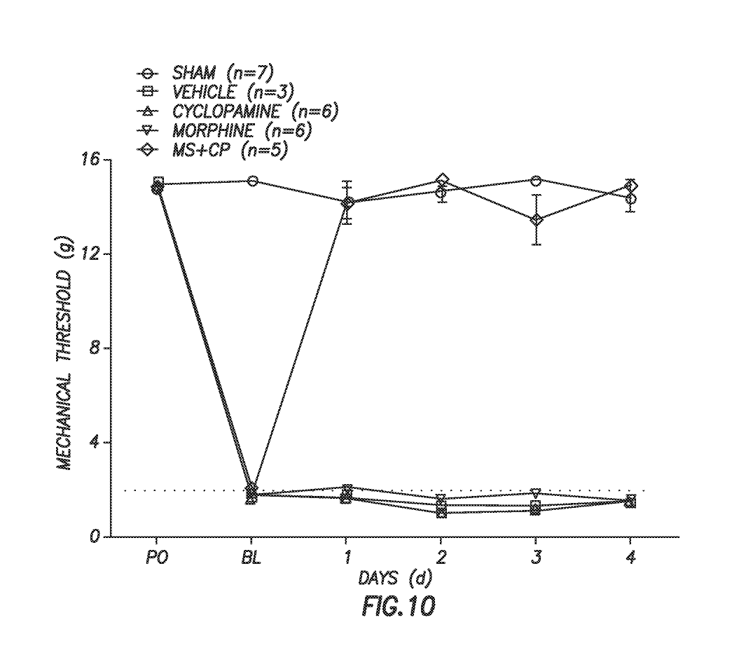

Over-expression of Patched (UAS-ptc) severely inhibited development of thermal allodynia 24 hours post UV (FIG. 10a). In control larvae (ppk1.9-Gal4 alone) about 70% of larvae exhibited aversive withdrawal, with about half of those responding in under 5 seconds. Expression of UAS-ptc and other Hh-interfering transgenes resulted in under 20% of larvae responding, almost all of which responded slowly (between 5 and 20 seconds). Interfering with Smoothened activity, using either tissue-specific RNA-interference (UAS-smo^{IR}) or expressing a dominant-negative form of the protein (UAS-smo.5A), also limited thermal allodynia. Collins, R. T. & Cohen, S. M., A Genetic Screen in Drosophila for Identifying Novel Components of the hedgehog Signaling Pathway, Genetics 170, 173-184 (2005). The development of allodynia involves a transcriptional component, as expression of a dominant-negative form of the transcription factor Cubitus Interruptus (UAS-ci⁷⁶) prevented allodynia. Aza-Blanc, P., et al., Proteolysis That is inhibited By Hedgehog Targets Cubitus Interruptus Protein to the Nucleus and Converts It to a Repressor, Cell 89, 1043-1053 (1997). Finally, canonical transcriptional targets of the Rh pathway, including engrailed and dpp, also played a role in thermal allodynia.

We tested larvae of these same genotypes for the development of thermal hyperalgesia eight hours after UV exposure, and found that in no case did the withdrawal latency drop as in control larvae (FIG. 10B). These data suggest that canonical Hh signaling within nociceptive sensory neurons is required for development of both thermal allodynia and hyperalgesia. As previously described and shown in FIGS. 1 through 4, inhibition of positive regulators of the Hh pathway (shown in FIG. 5) or overexpression or activation of negative regulators (shown in FIG. 5) leads to loss of development of tissue damage-induced allodynia and hyperalgesia. Thus, components of the Hedgehog signaling pathway represents a novel and completely unexpected set of potential drug targets for inhibiting the development of pain sensitization.

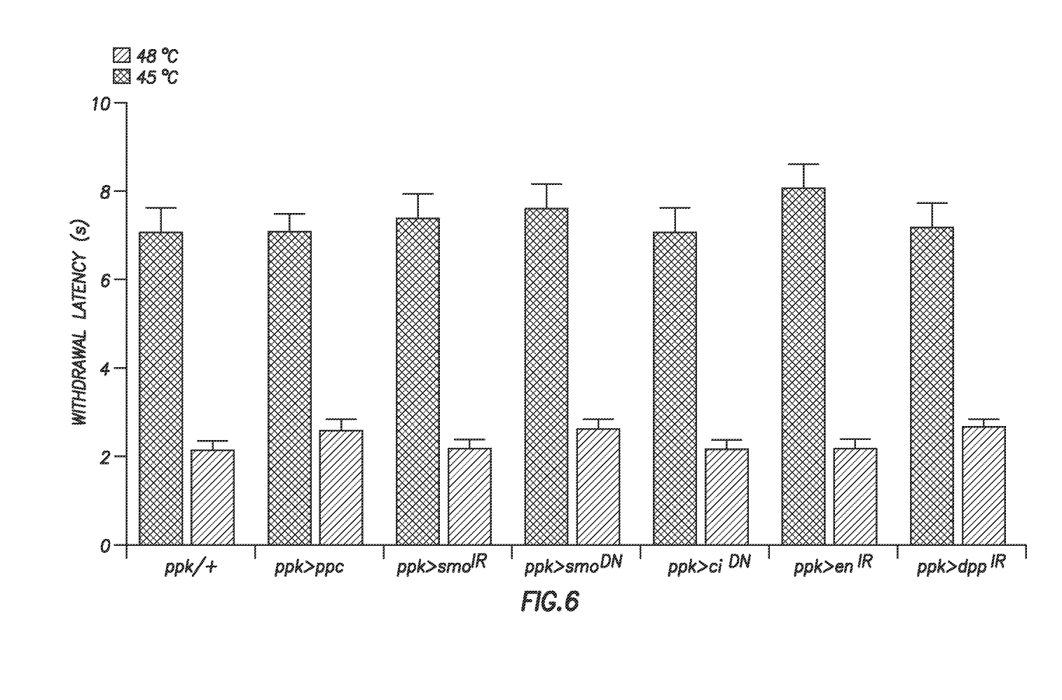

Baseline nociception is normal when Hh signaling is blocked. While Hh signaling is required for thermal allodynia and hyperalgesia, blocking the pathway had no effect on baseline nociception without UV treatment (FIG. 6s). In the absence of tissue damage, there were no significant impairments to withdrawal latency measured at 45° C., the lowest temperature at which all larvae exhibit aversive withdrawal within our 20 s cutoff, and 48° C., the lowest temperature at which all larvae exhibit aversive withdrawal within 5 seconds. Babcock. D. T. et al., Cytokine Signaling Mediates UV-Induced Nociceptive Sensitization in Drosophila Larvae, Curr Biol. 19, 799-806 (2009). Thus, Hh signaling in nociceptive sensory neurons mediates tissue damage-induced changes in the behavioral response threshold without affecting the baseline nociceptive threshold.

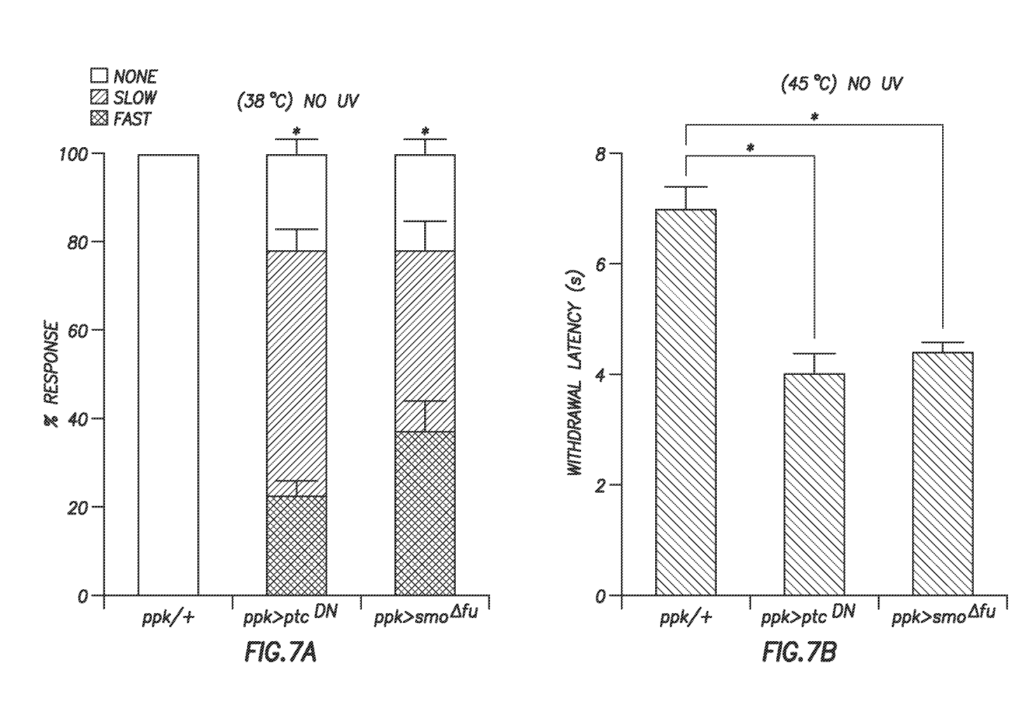

Ectopic activation of Hh signaling causes chronic nociceptive sensitization in the absence of tissue damage. In an important test of its sufficiency to mediate nociceptive sensitization, we found that Hh does not require other factors to be released from damaged cells to mediate its effects on nociceptive sensitization. We tested whether activation of the Hh pathway in nociceptive sensory neurons was sufficient to cause hypersensitivity in the absence of tissue damage. Constitutive activation of the pathway was achieved by expression of a dominant-negative form of the Hh repressor Patched (UAS-ptc^{1130X}) or a form of Smoothened that cannot interact with the downstream kinase, Fused (UAS-Smo^{Δfu})² Johnson, R. L. et al.,In Vivo Functions of the Patched Protein: Requirement of the C Terminus for Target Gene Inactivation But Not Hedgehog Sequestration, Mol. Cell 6, 467-478 (2000); Malpel, S. et al., The Last 59 Amino Acids Of Smoothened Cytoplasmic Tail Directly Bind the Protein Kinase Fused and Negatively Regulate the Hedgehog Pathway, Dev Biol 303, 121-133 (2007). In both cases, we found that non-irradiated larvae exhibited a robust response to a normally subthreshold stimulus of 38° C. (FIG. 7A) and displayed an exaggerated response to a normally noxious stimulus of 45° C. (FIG. 7B), Taken together, these results demonstrate that ectopic activation of the Hh signaling pathway can evoke thermal allodynia and hyperalgesia even in the absence of tissue damage. Furthermore, the larvae expressing either UAS-Smo^{Δfu} or UAS-ptc^{1130X} represent novel tools for the genetic identification of genes that act downstream of the Hh signaling cascade to mediate nociceptive sensitization.

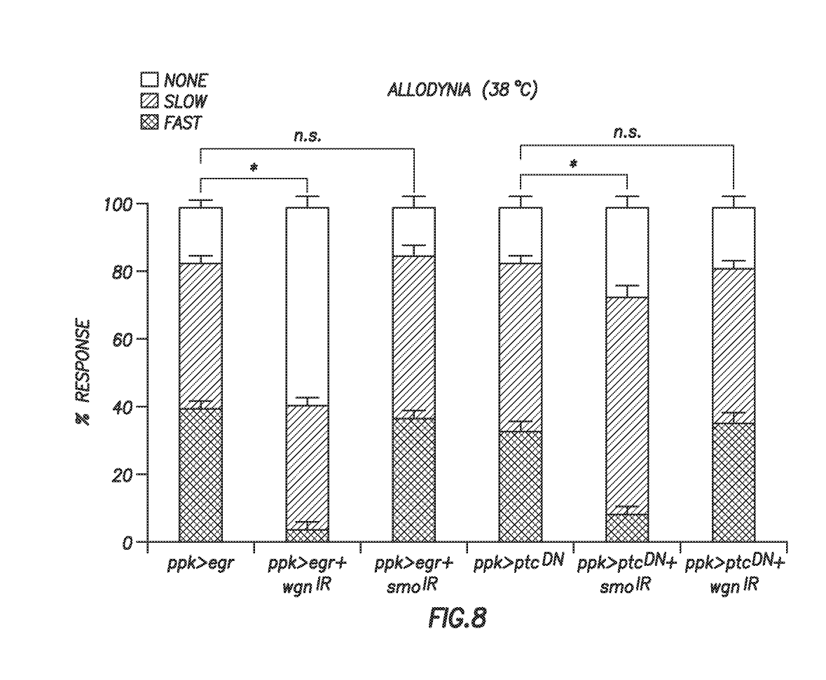

In Drosophila, Hedehog signaling acts independently of TNF signaling to mediate nociceptive sensitization. Our previous studies demonstrated that the Drosophila TNF ortholog, Eiger, and its receptor, Wengen, were required for thermal allodynia following UV damage. Babcock, D. T. et al., Cytokine Signaling Mediates UV-Induced Nociceptive Sensitization in Drosophila Larvae, Curr Biol 19, 799-806 (2009). We next performed genetic epistasis experiments to test whether Hh-mediated thermal allodynia depends on TNF signaling or vice versa. To determine this, we constitutively activated one pathway while simultaneously interfering with the other. Like ectopic activation of Hh signaling (FIG. 7), over-expression of Eiger/TNF in nociceptive sensory neurons induced thermal allodynia even in the absence of tissue damage. Id. As expected, blocking TNF signaling by knocking down nociceptive sensory neuron expression of Wengen dampened ectopic Eiger/TNF-induced thermal allodynia (FIG. 8). When Hh signaling is impaired via knockdown of Smoothened, however, the TNF-induced thermal allodynia was not affected, suggesting that Smoothened does not act downstream of Eiger/TNF in mediating thermal allodynia.

The vector, ppk1.9-Gal4 drives expression of the indicated UAS transgenes in nociceptive sensory neurons. Behavioral response to a normally non-noxious 38° C. stimulus upon activation of Hh (UAS-ptc^{1130X}=ptc^{DN}) or TNF (eiger^{GS9830}=egr) signaling in the presence or absence of transgenes blocking each pathway (UAS-wengen^{IR} to block TNF or UAS-smo^{IR} to block Hh). Constitutive activation of TNF signaling causes allodynia that is reduced by knockdown of Wengen, but not Smoothened. Constitutive activation of Hh signaling causes allodynia that is reduced by knockdown of Smoothened, but not Wenger. IR=Inverted Repeat. DN=Dominant Negative. N=triplicate sets of 30 larvae per condition. Error bars represent Standard Error of the Mean (S.E.M).

Constitutive activation of Hh signaling via a dominant-negative form of Patched (FIG. 7) caused UV-independent thermal allodynia that was dampened by knocking down expression of Smoothened (FIG. 8). However, when TNF signaling is blocked in the presence of Hh activation, Hh-induced thermal allodynia was not impaired, suggesting that TNF signaling is not downstream of Hh in mediating thermal allodynia. Taken together, these results show that TNF- and Hh-induced thermal allodynia in Drosophila operate by distinct and parallel mechanisms.

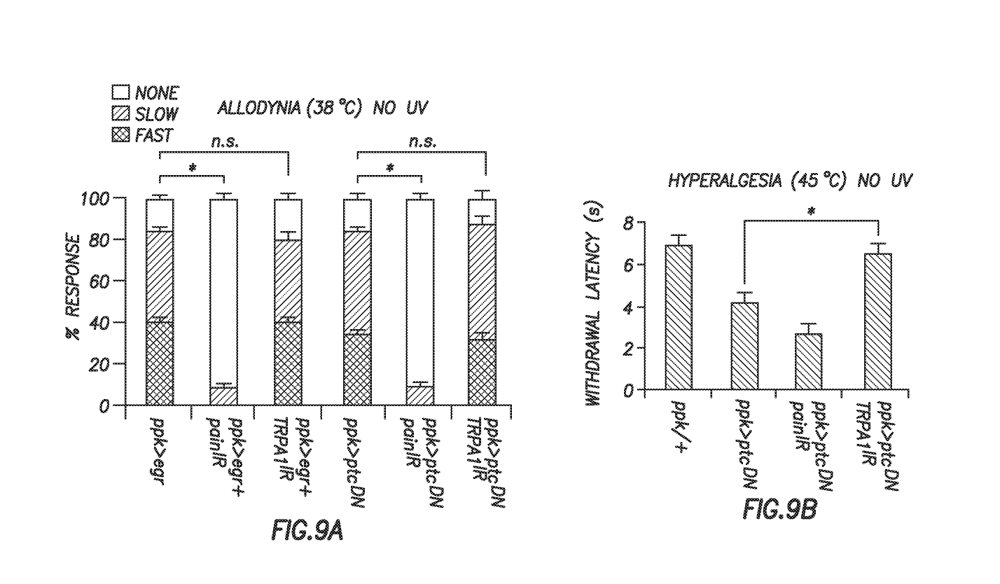

Hh and TNF-induced nociceptive sensitization require TRP channel activity. Members of the family of transient receptor potential (TRP) channels are required for detection of noxious thermal, mechanical and chemical stimuli in both Drosophila and vertebrates. Julius, D. et al., Molecular Mechanisms of Nociception, Nature 13:413(6852):203.-19 (2001). To determine whether nociceptive sensitization induced by either TNF or Hh signaling requires TRP channel activity, we crossed our constitutively sensitized lines (ppk1.9-Gal4+UAS-eiger for TNF activation and ppk1,9-Gal4+UAS-PatchedDN for Hh activation) to flies bearing UAS-RNAi lines that target TRP channels previously implicated in nociception in flies. Tracey, W. D., Jr., et al., Painless, A Drosophilia Gene Essential for Nociception, Cell 113:261-273, (2003); Kang, K. et al., Analysis of Drosophilia TRPA1 Reveals an Ancient Origin for Human Chemical Nociception. Nature 464: 597-600. FIG. 9A shows that knockdown of the Painless TRP channel blocks both TNF- and Hh-induced allodynia whereas knockdown of the TRPA1 channel does not. Tracey, W. D., et al., Painless, A Drosophilia Gene Essential for Nociception. Cell 113:261-273 (2003). FIG. 9B shows that Hh-induced hyperalgesia, by contrast, requires TRPA1 but not Painless. These results suggest that both TNF- and Hh-induced nociceptive sensitization act through the types of conserved ion channels that have previously been implicated in pain signaling in a variety of organisms.

Hh signaling modulates neuropathic pain, inflammatory pain, and opioid tolerance in rats. Having identified the Hh signaling pathway as a new pathway required for nociceptive sensitization in Drosophila, we next examined whether Hh signaling also plays a role in nociceptive sensitization in vertebrates, a critical first step in demonstrating that blockade of the pathway might have clinical utility in humans. As shown in FIGS. 10, 11 and 12, we examined the effect of the specific Smoothened inhibitor (cyclopamine) on neuropathic pain, inflammatory pain and opioid tolerance in rats. With regards to neuropathic pain, animals underwent sciatic nerve ligation as previously described in Chung et al., and were then allowed to recover for two weeks. Allodynia was measured by assessing the withdrawal threshold to a mechanical stimulus (Von Frey hairs). Preoperative thresholds were approximately 14 g, and remained at this level in sham-operated animals. Postoperative baseline thresholds were 3-4 grams. Drugs were administered using lumbar puncture under anesthesia. Neither a sub-clinical dose of morphine nor cyclopamine had analgesic effects when given individually, but the combination completely reversed pain sensitivity for an extended period (MS+CP-morphine and cyclopamine),

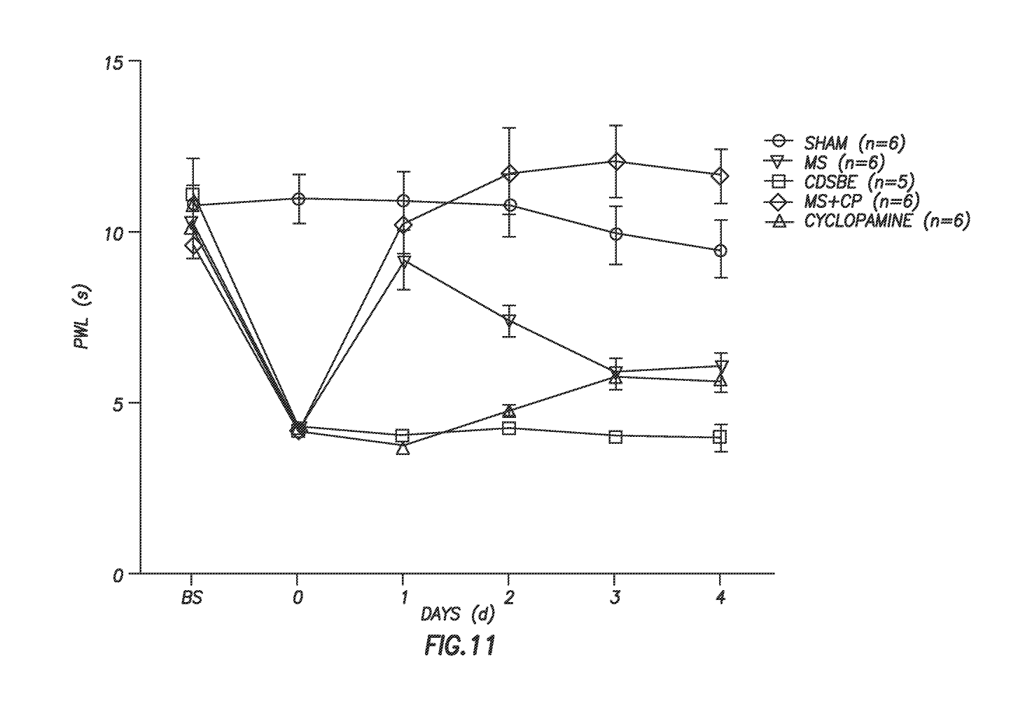

As shown in FIG. 11, animals underwent complete Freund's adjuvant (CFA) injection in the paw under anesthesia to induce local inflammatory nociceptive sensitization. Hyperalgesia was then measured using withdrawal latency of the paw to a thermal stimulus (PWL). Preadjuvant latencies were approximately 10-12 seconds, and remained at this level in sham-operated animals. Post-adjuvant baseline thresholds were 3-4 grams. Drugs were administered using lumbar puncture under anesthesia.. Morphine alone exhibited analgesic effects, but tolerance developed rapidly. By Day 3, cyclopamine developed a mild analgesic effect that plateaued. The combination of morphine and cyclopamine completely reversed pain hyperalgeisa for an extended period, without the development of tolerance (MS+CP-morphine and cyclopamine).

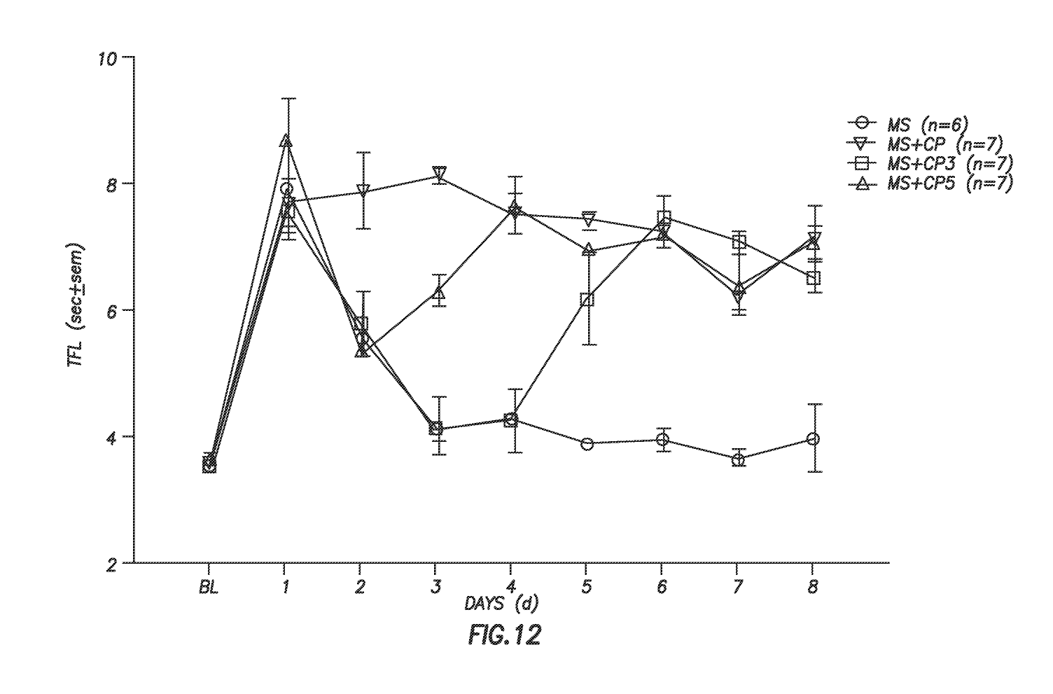

Drugs were administered daily using lumbar puncture under anesthesia. Tolerance was then assessed using tail flick latency (TFL), or the withdrawal latency of the tail to a thermal stimulus. Baseline (BL) latencies were 3-4 seconds. Animals received morphine alone (MS), morphine and cyclopamine (MS+CP), morphine alone with cyclopamine added on Day 3 (MS+CP3), or morphine alone with cyclopamine added on Day 5 (MS+CP5). On Day 8, all animals received morphine alone. Cyclopamine completely reversed tolerance within two days, even in animals that were completely tolerant (FIG. 12). The fact that animals were not tolerant on Day 8 after cyclopamine was removed indicates that it reversed the underlying biochemical processes responsible for the development of morphine tolerance. These findings could have profound implications for patients that are already extremely tolerant to opioids

The role of Hh signaling in modulation of nociceptive sensitization is conserved in mammals. To test this, we examined the effects of pharmacologically blocking Smoothened activity in rodent pain models. Injection of Complete Freund's Adjuvant (CFA) into the rat hindpaw causes local inflammation, swelling, and decreased nociceptive thresholds, Stein, C., et al., A. Unilateral inflammation of the Hindpaw in rats as a Model of Prolonged Noxious Stimulation: Alterations in Behavior and Nociceptive Thresholds, Pharmacol Biochem Behav 31, 455-451 (1988). The results are shown in FIGS. 10 and 11. FIG. 11 provides an inflammatory pain paradigm. Here, the hindpaws were injected with saline (sham, n=6), or CFA. Thermal analgesia was assessed using paw withdrawal latency (PWL) following intrathecal administration of 10% Captisol (Vehicle, n=5), cylcopamine (CP, n=6), morphine (MS, n=6), or cyclopamine and morphine (MS+CP, n=6). PI-Pre-injection of hindpaw; BL-baseline one day after paw injection, prior to intrathecal drug; n=number of animals per treatment; *−P<0.01 vs. vehicle. Similarly, FIG. 10 provides a neuropathic pain paradigm. Hence, rats underwent sciatic nerve ligation or sham (n=7) operation. Animals received daily intrathecal injections of vehicle (n=3) or drugs (CP, n=6; MS, n=6; or MS+CP, n=5). Mechanical allodynia was assessed using Von Frey filaments. PO-pre-operative; BL; baseline measurement 2 weeks after operation; n=number of animals per treatment; *—P<0,001 vs. vehicle. All data are+/−S.E.M.

After CFA injection, we observed a robust, sustained thermal hyperalgesia (FIG. 12A), where the paw withdrawal latency was decreased from 11.0 to 4.3 s up to 4 days post injection. Intrathecal administration of morphine caused analgesia, although tolerance developed with repeated dosing. Intrathecal administration of cyclopamine, a Smoothened inhibitor, had no analgesic effect. Chen, J. K., et al., Inhibition of Hedgehog Signaling by Direct Binding of Cyclopamine to Smoothened, Genes Dev 16, 2743-2748 (2002). Combining cyclopamine with morphine did not alter the acute analgesic effect of morphine (See, FIG. 11 and compare MS and MS+CP on Day 1), but surprisingly, blocked tolerance to the analgesic effect of morphine in this paradigm (FIG. 12A and compare MS and MS+CP on Days 2-4). Cyclopamine administration in a model of neuropathic pain produced similar results. In this model, neither cyclopamine nor a low dose of morphine had analgesic effects alone. However, the combination of morphine and cyclopamine caused complete and sustained reversal of mechanical allodynia (FIG. 10). Chung, J. M., et al., Segmental Spinal Nerve Ligation Model of Neuropathic Pain, Methods Mol. Med. 99, 35-45 (2004). Taken together, these results suggest that in mammals Smo signaling exerts its effects on nociceptive sensitivity through mechanisms that intersect with opioid receptor signaling.

Hh signaling has established roles in patterning, axon guidance, and proliferation during neural development. However, other than cancer and other proliferative responses. Hh has not been implicated in the physiological functions of differentiated neurons. Amankulor, N. M. et al. Sonic Hedgehog Pathway Activation Is Induced By Acute Brain Injury and Regulated By Injury-Related Inflammation, J Neurosci 29, 10299-10308 (2009); Goodrich, L. V., et al., Altered Neural Cell Fates and Medulloblastoma in Mouse Patched Mutants, Science 277, 1109-1113 (1997). Our results identify canonical Hh signaling as a major new pathway in modulation of nociception. How Hh alters the behavioral response threshold of nociceptive sensory neurons is not yet known. However, in flies it appears to be distinct from the mechanism employed by Eiger/TNF, as these pathways can independently cause thermal allodynia while only Hh can mediate thermal hyperalgesia. Hence, Allodynia and hyperalgesia appear to be genetically separable processes, Babcock, D. T. & Galko, M. J., Two Sides of the Same Coin No Longer: Genetic Separation of Nociceptive Sensitization Responses, Communicative & Integrative Biology 2, 58-60 (2009).

We have discovered that ectopic activation of the Hedgehog signaling pathway in nociceptive sensory neurons leads to constitutive pain sensitization in the absence of tissue damage. Genetic targeting of Hedgehog signaling components, including classical Hh alleles and RNAi inhibition of Smoothened, can block allodynia.

Cyclopamine (and analogs thereof) can act as an Hh signaling antagonist, and can reverse the development of opioid tolerance and treat neuropathic and inflammatory pain in rats. Cyclopamine reverses the development of morphine tolerance, which has extremely important implications for addiction as well as the treatment of patients that are already tolerant to opioids. In pain perception as in its developmental functions, the architecture and action of the signaling pathway is conserved between invertebrates and vertebrates. Pharmacological interference with the Hh pathway can be used to block the nociceptive sensitization that occurs in trauma, post-surgical recovery, and certain types of cancer pain and chemotherapy.

Intrathecal administration of one or more inhibitors of Sonic Hedgehog signaling can also block the development of analgesic tolerance to morphine in inflammatory pain, and if provided on a daily basis, morphine analgesia in neuropathic pain. Furthermore, the knockdown of Smoothened does not affect baseline pain sensation-only pain sensitization. A dominant-negative allele of Smoothened phenocopies the RNAi result and it blocks development of allodynia This allele can be target specific. Smoothened is also required for development of hyperalgesia following UV irradiation-see FIGS. 4 & 5. Pharmacological blockade of Smoothened signaling in vertebrates (rats) is shown here to interfere with development of allodynia and hyperalgesia, in both inflammatory and neuropathic pain.

Other Hedgehog pathway genes such as the Hh ligand itself, engrailed, the Gli-like transcription factor Cubitus interuptus, and its transcriptional cofactor, are also associated with the development of allodynia. See, FIG. 3. Components of the Hedgehog signaling pathway thus represent a new set of potential drug targets for treatment of pain, an enormous clinical problem in a variety of settings.

Because a role for Hh in modulation of nociceptive hypersensitivity is conserved in mammals, our results establish the power of using model genetic organisms to identify important new pathways in nociceptive biology. In rats, blocking Hh signaling prevents the development of morphine analgesic tolerance in a model of inflammatory pain and provides synergistic, sustained analgesia when combined with a subanalgesic dose of morphine in a neuropathic pain paradigm. These findings indicate an interaction between Hh and opioid signaling in mammals and also suggest that Hit signaling components represent a major new set of therapeutic targets for clinical pain treatment.

Small molecule inhibitors that inhibit Sonic Hedgehog signally and target Smo include cyclopamine; KAAD-cyclopamine, jervine. SANT1, SANT2, SANT3, SANT4, Cur-61414, IPI-926 and GDC-0449, Stanton, Benjamin et al, Small-Molecule Modulators of the Sonic Hedgehog Signaling Pathway, Mol. BioSyst., 6, 44-55 (2010), wherein FIG. 1 on page 45, FIG. 2 on page 49, FIGS. 3, 4, and 5 on page 51, FIG. 6 on page 52, and Table 2 on page 50 are incorporated herein by reference. In addition, Robotnikinin binds directly to Sonic Hedgehog. Id.

Table 1, immediately below, is a comparison of the Hedgehog Pathway in Drosophila Melanogaster and Mammals provided by Hooper, J. E., & Scott, M. P., Communicating with Hedgehogs, Nat. Rev. Mol, Cell Biol, 6, 306-317 (2005), Table 1, incorporated herein by reference.

TABLE 1

| | D. Melanogaster | Mammals |
|---|---|---|
| Ligands | Hh | SHH, DHH, IHH |
| Acyl transferase | Rasp | SKN |
| Maturase | Disp | DISPA |
| Receptors | Ptc | Ptc1, PTC2 |
| Glypican | Dlp | ? |
| Other Hh-blinding | Shf, Pxb? | HlP, GAS1, megalin |
| factors | | |
| Iniator | Smc | SMO |
| Smo regulartors | PKA, CKI, GSK3β | β-arrestin-2, GRK2 |
| Cystoplasmic | Cos2 | KIF7, KIF3a, IFT88, IFT172, |
| regulators | | RAB23, MIM/BEG4, FKPB8, |
| | | SIL |
| Cytoplasmic | Fu, Sufu | FU(?), SUFU iguaria |
| regulators | | |
| Transcription factors | Ci | GI I1, GI I2, and GI I3 |

Table 2 immediately below provides a list of current pharmacological inhibitors of the vertebrate Hh signaling pathway which may be used in the methods of treating pain described herein.

TABLE 2

| Small Molecule | Effects |
|---|---|
| Cyclopamine | Targets Smo |
| | Inhibits transcription of Gli1, Gli2, PtchI and other Shh-target |
| | genes in a variety of cell types |
| Jervine | Targets Smo |
| | Inhibits transcription of Gli1, Gli2, PtchI and other Shh-target |
| | genes in a variety of cell types |
| Staurosporinone | Inhibits Glil,2-mediated transcription in HaCat cells |
| | Inhibits transcription of Ptchl and BCL2 in HaCaT cells |
| Zerumbone | Inhibits Glil,2-mediated transcription in HaCat cells |
| | Inhibits transcription of Ptchl and BCL2 in HaCaT cells |
| Physalin B | Inhibits Glil,2-mediated transcription in HaCat cells |
| | Inhibits transcription of Ptchl and BCL2 in HaCaT cells |
| Physalin F | Inhibits Glil,2-mediated transcription in HaCat cells |
| | Inhibits transcription of Ptchl and BCL2 in HaCaT cells |
| 20α-Hydroxycholesterol | Does not bind directly to Smo |
| | Activates Gli-mediated transcription in a variety of cell types |
| 22(S)-Hydroxycholesterol | Does not bind directly to Smo |
| | Activates Gli-mediated transcription in a variety of cell types |
| 24(S)-Hydroxycholesterol | Does not bind directly to Smo |
| | Activates Gli-mediated transcription in a variety of cell types |
| 25-Hydroxycholesterol | Does not bind directly to Smo |
| | Activates Gli-mediated transcription in a variety of cell types |
| SANT1,2,3,4 | Targets Smo |
| | Represses Gli-medicated transcription in a variety of cell types |
| Cur-61414 | Represses Gli-medicated transcription in a variety of cell and |
| | tissues types |
| | Targets Smo |
| GANT58 | Represses Gli-medicated transcription in a variety of cell types |
| | Acts downstream of Smo and Sufu |
| GANT61 | Represses Gli-medicated transcription in a variety of cell types |
| | Acts downstream of Smo and Sufu |
| Robotnikinin | Represses Gli-medicated transcription in a variety of cell types and |
| | synthetic human tissue |
| | Binds directly to Shh |
| SAG | Activates Gli-mediated transcription in a variety of cell types |
| | Binds Smo and competes directly with cyclopamine-Smo |
| | interactions. |
| Purmorphamine | Activates Gli-mediated transcription in a variety of cell types |
| | Binds Smo and competes directly with cyclopamine-Smo |
| | interactions. |
| IPI-926 | Represses Gli-medicated transcription in a variety of cell types |
| | Targets Smo |
| | Clinical trials for BCC and pancreatic adenocarcinoma are ongoing |
| GDC-0449 | Represses Gli-medicated transcription in a variety of cell types |
| | Targets Smo |
| | Clinical trials for BCC and other solid tumors are ongoing |

Stanton, B, et al., Small-Molecule Modulators of the Sonic Hedgehog Signaling Pathway, Mol. BioSyst. 44:6, 44-54 (2010), Table 2 at 50, incorporated herein by reference.

Methods

Fly Stocks and Genetics

Almost all fly stocks were maintained at 25° C. The exceptions were the temperature-sensitive allele of hh (hh^{ts2}) and paired w¹¹¹⁸ controls, which were kept at 18° C. (permissive temperature) and shifted to 29° C. (restrictive temperature) under some experimental conditions (See FIG. 9). Ma, C., et al., The Segment Polarity Gene Hedgehog is Required For Progression of the Morphogenetic Furrow in the Developing Drosophila Eye, Cell 75, 927-938 (1993). For heat-shock experiments, UV- or mock-treated larvae were placed at either the permissive or restrictive temperature until assessment of thermal hyperalgesia (8 hour, 45° C.) or thermal allodynia (24 hour, 38° C.).

We used the GAL4/UAS system to drive expression of UAS transgenes in larval nociceptive sensory neurons (ppk1.9-Gal4), UAS-smo.5A (=UAS-smo^{DN}), UAS-ptc, UAS-ci⁷⁶(=UAS-ci^{DN}) and UAS-dpp^{IR} were used to inhibit Hh signaling, along with the following UAS-RNAi lines: 11561 (9542) targeting Smoothened; and 9015 (105678) targeting Engrailed. UAS-ptc^{1130X} (=UAS-ptc^{DN})⁾ and UAS-Smo^{Δfu} were used to constitutively activate Hh signaling. Ainsley, J. A. et al, Enhanced Locomotion Caused by Loss of the i Drosophila DEG/ENαC protein Pickpocket1, Curr Biol 13, 1557-1563 (2003); Collins, R. T. & Cohen, S. M., A Genetic Screen in Drosophila for identifying Novel Components of the Hedgehog Signaling Pathway, Genetics 170, 173-184 (2005); Aza-Blanc, P., et al., Proteolysis That is Inhibited by Hedgehog Targets Cubitus Interruptus Protein to the Nucleus and Converts it to a Repressor, Cell 89, 1043-1053 (1997); Johnson, R. L., et al., In viva Functions of the Patched Protein: Requirement of the C Terminus for Target Gene Inactivation but not Hedgehog Sequestration, Mol Cell 6, 467-478 (2000); Malpel, S. et al. The Last 59 Amino Acids of Smoothened Cytoplasmic Tail Directly Bind the Protein Kinase Fused and Negatively Regulate the Hedgehog Pathway, Dev Biol 303, 121-133 (2007); Brand, A. H. & Perrimon, N., Targeted Gene Expression as a Means of Altering Cell Fates and Generating Dominant Phenotypes, Development 118, 401-415 (1993); Ni, J. Q. et al., Vector and Parameters For Targeted Transgenic RNA Interference In Drosophila Melanogaster, Nat Methods 5, 49-51 (2008); Dietzl, G. et at, A Genome-Wide Transgenic RNAi Library For Conditional Gene inactivation In Drosophila, Nature 448, 151-156 (2007); Igaki, T. et al., Eiger, a TAT Superfamily Ligand that Triggers the Drosophila JNK Pathway, Embo J 21, 3009-3018 (2002),

The UAS-bearing EP allele eiger^{GS9830} was used to overexpress eiger. UAS-wengen^{IR} was used to inhibit TNF signaling. Igaki, T. et al., Eiger, α TNF Superfamily Ligand that Triggers the Drosophila JNK Pathway, Embo J 21, 3009-3018 (2002); Kanda, H., et al., Wengen, A Member of the Drosophila Tumor Necrosis Factor Receptor Superfamily is Required For Eiger Signaling, J Biol Chem 277, 28372-28375 (2002).

UV Treatment and Assessment of Nociceptive Behavior (Drosophila)

UV radiation of early third instar larvae was carried out as previously reported. Babcock, D. T. et al., Cytokine Signaling Mediates UV-Induced Nociceptive Sensitization in Drosophila Larvae, Curr Biol 19, 799-806 (2009). Noxious and non-noxious thermal stimuli were delivered using a custom-built heat probe. Id. Stimuli were presented along the dorsal midline in abdominal segments A4-A6. The withdrawal latency to each stimulus was recorded up to a 20 seconds cutoff. The withdrawal behavior is defined as at least one complete 360° roll in response to the stimulus. We tested for thermal allodynia at 38° C., the highest temperature at which control larvae do not respond in the absence of tissue damage. We tested for thermal hyperalgesia at 45° C., the lowest noxious temperature at which all control larvae respond with aversive withdrawal. Allodynia was assessed 24 hour post UV and hyperalgesia 8 hour post UV, the time at which these responses peak in control larvae.

Vertebrate Neuropathic and Inflammatory Nociception Assays

Animals: Sprague-Dawley rats (male, 250-300 mg) were housed three to a cage with water and food ad libitum and kept in temperature controlled rooms on a 12:12 hour light-dark cycle, with the dark cycle beginning at 7:00 pm. Animals were habituated to the testing environment for one week prior to testing and all tests were performed in the morning. All protocols were approved by our Institutional Animal Care and Use Committee.

Drugs and Intermittent Lumbar Puncture: Rats were anesthetized with 2% isoflurane in oxygen. The lumbar region was shaved, prepared with Betadine solution and the intervertebral spaces widened by placing the animal on a plexiglas tube. Animals then underwent lumbar puncture daily at the L 5-6 interspace as previously described, using a ½″ 30-gauge needle connected to a Hamilton syringe filled with 10% Captisol (Cydex; Lenexa, Kans.) vehicle, or drugs dissolved in Captisol: 0.4 nmol morphine sulfate (Mallinckrodt, Inc., St. Louis, Mo., USA), 10 μg cyclopamine (LG Laboratories, Woburn, Mass., USA, or the combination of morphine and cyclopamine. REF 35. Correct subarachnoid positioning of the tip of the needle was verified by a tail or/and paw flick. Animals recovered in their home cage for 45 minutes prior to analgesic testing.

Complete Freund's Adjuvant (CFA) infection: Under isoflurane analgesia, the plantar surface of the left hindpaw was cleansed and 100 μl of CFA (Sigma, St. Louis, Mo.) injected using a 25-gauge needle. Animals recovered for 24 hours prior to analgesic testing.

Segmental Spinal Nerve Ligation (SNL): Under isoflurane anesthesia, rats underwent segmental ligation of the left L5 nerve root as previously described. Animals were allowed to recover for two weeks before mechanical latency testing or drug administration was performed.

Paw Withdrawal Latency (PWL): PWL was measured by placing the animals in Plexiglas cages (9×22×25 cm.) on a modified Hargreaves Device, consisting of a glass surface maintained at 30° C. A stimulus lamp was focused on the tail with three measurements taken on different portions of the tail. An abrupt flick of the tail secondary to the thermal stimulus was sensed by photodiodes, and this served to terminate the stimulus and stop the timer. Animals were habituated to the device for one week prior to testing and for 30 min before each test session. The intensity of the thermal stimulus used in the TFL test was adjusted so baseline TFL was between 10-12 seconds to 20 second was used as an automatic cutoff time to avoid paw damage. The TFLs at the time points of 15, 30, and 45 minutes post-injection were measured and compared (n=6-8 per group).

Mechanical Latency Threshold: Animals were habituated to a plexiglas cage with wire mesh floor as described above. Animals were then tested using graded von Frey filaments (Stoelting Corporation, Wood Dale, Ill., USA) in ascending stiffness.

Statistical Analysis

For Drosophila nociception assays, we used Fisher's exact test to compare categorical responses: no response within 20 second cutoff; slow response (aversive withdrawal between 5 and 20 second); or fast response (aversive withdrawal in under 5 seconds). When the mean withdrawal latency is compared among genotypes, we used the Student's t-test or one-way analysis of variance (ANOVA). For rat nociception assays, results were reported as mean±S.E.M. Between-group comparisons were made by analysis of variance (ANOVA) and Bonferroni post-hoc tests. Differences were considered to be significant at P<0.05.

Flies were also produced to specifically express either dominant-negative Patched transgene or constitutively active Smoothened transgene in larval nociceptive sensory neurons. These larvae exhibit nociceptive sensitization even in the absence of tissue damage. The data from the flies is provided in FIG. 10. In all panels ppk1.9-Gal4 drives (>) expression of the indicated UAS transgenes or no transgene (ppk1.9-Gal4×w¹¹¹⁸=ppk/+) in nociceptive sensory neurons. As shown in FIG. 10(a), behavioral responses of larvae of indicated genotypes to a stimulus of 38° C., 24 h after UV treatment. N=triplicate sets of 30 larvae per condition. FIG. 10(b) shows the response data of larvae of indicated genotypes to a 45° C. stimulus without UV and 8 h after UV treatment and N=50 larvae in FIG. 10(c), the baseline nociception in response to a 45° C. or 48° C. stimulus in the absence of UV damage. N=50 larvae. FIGS. 10(d) and FIG. 10(e) show the constitutive activation of Hh signaling in the absence of UV irradiation produced thermal allodynia to a 38° C. stimulus (FIG. 10(d)) and thermal hyperalgesia to a 45° C. stimulus (e). IR=Inverted Repeat. DN=Dominant Negative. N=triplicate sets of 30 larvae per condition. Error bars represent Standard Error of the Mean (S.E.M). Hh-mediated sensitization acts through certain TRP channels in the sensitized larvae.

### EXAMPLE 1

### Formulation and Administration of Cyclopamine, an Inhibitor of Smoothened

Rats were anesthetized with 2% isoflurane in oxygen via nose cone. The lumbar region was shaved, prepared with Betadine solution, and the intervertebral spaces widened by placing the animal on a plexiglas tube. Animals were then injected at the 15-6 interspace using a 0 5-inch 30-gauge needle (Becton-Dickinson, Franklin Lakes, N.J.) connected to a Hamilton syringe filled with morphine (morphine sulfate; Mallinckrodt Inc, Si Louis, Mo.) and cyclopamine were dissolved in 20 μl of a solution of artificial cerebrospinal fluid (aCSF 126 mM of NaCl, 2.5 mM of KCl, 1.2 mM of NaH2PO₄, 1.2 mM of MgCl₂, 2.4 mM of CaCl₂, 11 mM of glucose, and 25 mM of NaHCO₃, saturated with 5% CO₂ in 95% O₂, and adjusted to a pH value of 7.3-7.4) and 10% final concentration (w/v) sulfobutylether-7-b-cyciodextrin (Captisol, Cydex, Lenexa, Kans.). Correct subarachnoid positioning of the tip of the needle was verified by a tail- or paw-flick test. Animals then recovered in their home cage before analgesic testing. For subcutaneous administration, morphine sulfate and cyclopamine were dissolved in a solution of normal saline containing 10% Captisol, and injected subcutaneously in a volume of 1 ml/kg body weight.

We claim:

1. A method of treating nociception comprising the step of administering to a subject in need thereof a therapeutic amount of a compound that modulates the Hedgehog signaling pathway.

2. The method of claim 1, wherein morphine is administered to the subject in combination with the compound that modulates the Hedgehog signaling pathway.

3. A method of treating allodynia comprising the step of administering to a subject in need thereof a therapeutic amount of a compound that modulates the Hedgehog signaling pathway.

4. The method of claim 1, wherein morphine is administered to the subject in combination with the compound that modulates the Hedgehog signaling pathway.

5. A method of treating hyperalgesia comprising the step of administering to a subject in need thereof a therapeutic amount of a compound that modulates the Hedgehog signaling pathway.

6. The method of claim 1, wherein morphine is administered to the subject in combination with the compound that modulates the Hedgehog signaling pathway.

7. A method of reducing tolerance to opioid analgesics comprising the step of administering a therapeutic amount of cyclopamine to a subject in need thereof in combination with a therapeutic amount of opioid analgesic.

8. A transgenic fly comprising Drosphilia either dominant-negative Patched transgene, or constitutively active Smoothened transgene in larval nociceptive sensory neurons.


SEQUENCE LISTING (2 sequences)

SEQ ID NO: 1 - DNA, 3111 bp, Drosophila melanogaster
atgcagtacttaaactttccgcgcatgccaaacattatgatgttcctggaggttgcgatcttatgcctgtgggtggtcgcagacgcatcggccagttcggccaagttcggcagcacaacgcccgcaagtgcgcagcagtcggatgtggaactggagcccatcaatgggactctcaattaccgactgtacgccaagaagggcagggacgacaaaccctggtttgatggcctagacagcaggcacatccagtgtgtccgacgtgcccgttgctaccccacctcgaacgcaaccaacacctgtttcggctcaaaattgccctatgagctgagcagcctagatctcaccgacttccacaccgaaaaggagctgaacgataagctgaacgactactatgccctgaagcacgtgcccaaatgttgggcagctatacagccctttttgtgcgccgtctttaagccgaagtgtgaaaaaatcaacggcgaggacatggtctacctgccatcttacgagatgtgccgaattaccatggaaccctgtcgcattttgtacaacacgacgtttttcccaaaattccttcgctgcaacgaaacactctttccgacgaaatgcacaaacggagcacgaggaatgaaattcaacggaactggccagtgtttaagtcctttggttccgacagatacgtcagccagctattatcctggcatcgagggctgcggcgtgcgatgcaaggatccactctataccgatgatgagcatcggcagatccacaaactgatcggatgggctggcagcatatgtcttctgtctaaccttttcgtggtgtccaccttcttcatcgactggaagaatgccaacaagtatccggcagtaattgtgttctacataaatctttgctttctaattgcttgcgtcggctggttgcttcagtttacttctggctcgcgagaggacatagtatgtcgtaaggatggaacacttcgccactcagagcctacagccggtgaaaatctttcttgcatagtgatctttgtgctggtctattattttctcaccgctggaatggtttggtttgtgttcctcacctacgcctggcattggagggccatgggccacgtccaagatcggatagataagaaaggttcctactttcacctcgtggcgtggtcactaccccttgtgcttaccattaccacgatggctttcagtgaggtggatggaaatagtattgtgggcatctgcttcgtaggctatatcaatcattctatgagggcaggactacttcttggtccgctctgcggggtcatcctcattggtggatacttcatcacccgcggcatggtgatgctttttggactgaaacacttcgctaatgacattaaatcaacttcggcgagcaacaaaatccatttgatcatcatgcgcatgggagtctgtgctctgctcactttagttttcatactagtggccattgcgtgccacgttacggagtttaggcatgcagacgaatgggcccagagcttcagacagtttataatctgcaaaatttcttcagtttttgaagaaaagagttcctgtcgaattgaaaaccgacctagtgttggcgttcttcaattgcatttgctgtgtctatttagctctggaatcgtaatgtccacctggtgctggacaccttcttcaattgagacttggaagcgttatataaggaaaaagtgtggcaaagaggtggtcgaagaagtgaaaatgccgaagcacaaggtcattgcccagacatgggccaagcgcaaggatttcgaggacaagggcaggctctccataacgctctacaacacccacacagatcccgtggggctcaacttcgatgtgaacgatctgaactcttctgagacgaatgacatctcatcaacttgggctgcatacctcccgcagtgcgtaaaacgtcgcatggctttgacgggagcagcgacaggtaactcgtcaagccatggaccgcgaaaaaattcattggattccgagataagtgtgagtgttcgacatgtttccgttgaatcccgcagaaattcggtggactcgcaggtatcagtgaaaatagctgaaatgaagaccaaagtggcgtccagatcaaggggaaaacacggaggctcttccagcaacagaagaacccaaaggagaagggattatatagcagctgccactggaaaaagcagtaggagaagggaaagcagtacttcagtggagtcgcaggtcatcgcgctcaagaaaacgacctatcccaatgctagtcacaaagtgggcgtgtttgctcatcacagctccaagaaacaacacaattacaccagctccatgaagcgaaggactgctaatgccggattggatccctctattcttaatgaattcctgcagaaaaatggcgattttatattcccattcctccaaaatcaagatatgagctctagttcggaggaggataattccagagcatcccaaaagattcaggatcttaacgtggttgtaaagcagcaggaaataagtgaggatgatcacgacggaataaagattgaagaactgccaaatagcaaacaggtggcattggagaactttcttaaaaacataaaaaaatctaatgaatccaattctaaccgacattcccgaaattccgcaagaagtcagtcaaaaaagtcccaaaagagacatctcaagaaccctgctgctgatctagatttcaggaaggactgtgtaaagtatcggtctaatgactcacttagctgctcctctgaagagctggatgtggctttggacgtaggaagccttcttaacagctctttttctggaatatccatgggcaaaccacatagtagaaacagcaaaaccagctgcgatgtgggcatacaggctaatcctttcgagctagttcccagttacggagaagacgaactgcagcaggccatgcgactcctaaacgcagccagcagacaaagaactgaagcagccaatgaggatttcggaggaacggagctgcagggcttgttgggtcattcccatcggcatcaaagggagcccacgtttatgagcgagtcggacaaactcaaaatgttattgctgccttcaaaatag

SEQ ID NO: 2 - DNA, 3861 bp, Drosophila melanogaster
atggaccgcgacagcctcccacgcgttccggacacacacggcgatgtggtcgatgagaaattattctcggatctttacatacgcaccagctgggtggacgcccaagtggcgctcgatcagatagataagggcaaagcgcgtggcagccgcacggcgatctatctgcgatcagtattccagtcccacctcgaaaccctcggcagctccgtgcaaaagcacgcgggcaaggtgctattcgtggctatcctggtgctgagcaccttctgcgtcggcctgaagagcgcccagatccactccaaggtgcaccagctgtggatccaggagggcggccggctggaggcggaactggcctacacacagaagacgatcggcgaggacgagtcggccacgcatcagctgctcattcagacgacccacgacccgaacgcctccgtcctgcatccgcaggcgctgcttgcccacctggaggtcctggtcaaggccaccgccgtcaaggtgcacctctacgacaccgaatgggggctgcgcgacatgtgcaacatgccgagcacgccctccttcgagggcatctactacatcgagcagatcctgcgccacctcattccgtgctcgatcatcacgccgctggactgtttctgggagggaagccagctgttgggtccggaatcagcggtcgttataccaggcctcaaccaacgactcctgtggaccacactgaatcccgcctctgtgatgcagtatatgaagcagaagatgtccgaggaaaagatcagcttcgacttcgagaccgtggagcagtacatgaagcgtgcggccattggcagtggctacatggagaagccctgcctgaacccactgaatcccaattgcccggacacggcaccgaacaagaacagcacccagccgccggatgtgggagccatcctgtccggaggctgctacggttatgccgcgaagcatatgcactggccggaggaactgattgtgggcggagcgaagaggaaccgcagcggacacttgaggaaggcccaggccctgcagtcggtggtgcagctgatgaccgagaaggaaatgtacgaccagtggcaggacaactacaaggtgcaccatcttggatggacgcaggagaaggcagcggaggttttgaacgcctggcagcgcaacttttcgcgggaggtggaacagctgctacgtaaacagtcgagaattgccaccaactacgatatctacgtgttcagctcggctgcactggatgacatcctggccaagttctcccatcccagcgccttgtccattgtcatcggcgtggccgtcaccgttttgtatgccttctgcacgctcctccgctggagggaccccgtccgtggacagagcagtgtcggcgtggccggagttctgctcatgtgctttagtaccgccgccggattgggattgtcagccctgctcggtatcgttttcaatgccgccagcacccaggtggttccgtttttggcccttggtctgggcgtcgatcacatcttcatgctgaccgctgcctatgcggagagcaatcggcgggagcagaccaagctgattctcaagaaagtgggaccgagcatcctgttcagtgcctgcagcacggcaggatccttctttgcggccgcctttattccggtgccggctttgaaggtattctgtctgcaggctgccatcgtaatgtgctccaatttggcagcggctctattggtttttccggccatgatttcgttggatctacggagacgtaccgccggcagggcggacatcttctgctgctgttttccggtgtggaaggaacagccgaaggtggcaccaccggtgctgccgctgaacaacaacaacgggcgcggggcccggcatccgaagagctgcaacaacaacagggtgccgctgcccgcccagaatcctctgctggaacagagggcagacatccctgggagcagtcactcactggcgtccttctctctggcaaccttcgcctttcagcactacactcccttcctcatgcgcagctgggtgaagttcctgaccgttatgggtttcctggcggccctcatatccagcttgtatgcctccacgcgccttcaggatggcctggacattattgatctggtgcccaaggacagcaacgagcacaagttcctggatgctcaaactcggctctttggcttctacagcatgtatgcggttacccagggcaactttgaatatcccacccagcagcagttgctcagggactaccatgattcctttgtgcgggtgccacatgtgatcaagaatgataatggtggactgccggacttctggctgctgctcttcagcgagtggctgggtaatctgcaaaagatattcgacgaggaataccgcgacggacggctgaccaaggagtgctggttcccaaacgccagcagcgatgccatcctggcctacaagctaatcgtgcaaaccggccatgtggacaaccccgtggacaaggaactggtgctcaccaatcgcctggtcaacagcgatggcatcatcaaccaacgcgccttctacaactatctgtcggcatgggccaccaacgacgtcttcgcctacggagcttctcagggcaaattgtatccggaaccgcgccagtattttcaccaacccaacgagtacgatcttaagatacccaagagtctgccattggtctacgctcagatgcccttttacctccacggactaacagatacctcgcagatcaagaccctgataggtcatattcgcgacctgagcgtcaagtacgagggcttcggcctgcccaactatccatcgggcattcccttcatcttctgggagcagtacatgaccctgcgctcctcactggccatgatcctggcctgcgtgctactcgccgccctggtgctggtctccctgctcctgctctccgtttgggccgccgttctcgtgatcctcagcgttctggcctcgctggcccagatctttggggccatgactctgctgggcatcaaactctcggccattccggcagtcatactcatcctcagcgtgggcatgatgctgtgcttcaatgtgctgatatcactgggcttcatgacatccgttggcaaccgacagcgccgcgtccagctgagcatgcagatgtccctgggaccacttgtccacggcatgctgacctccggagtggccgtgttcatgctctccacgtcgccctttgagtttgtgatccggcacttctgctggcttctgctggtggtcttatgcgttggcgcctgcaacagccttttggtgttccccatcctactgagcatggtgggaccggaggcggagctggtgccgctggagcatccagaccgcatatccacgccctctccgctgcccgtgcgcagcagcaagagatcgggcaaatcctatgtggtgcagggatcgcgatcctcgcgaggcagctgccagaagtcgcatcaccaccaccacaaagaccttaatgatccatcgctgacgacgatcaccgaggagccgcagtcgtggaagtccagcaactcgtccatccagatgcccaatgattggacctaccagccgcgggaacagcgacccgcctcctacgcggccccgccccccgcctatcacaaggccgccgcccagcagcaccaccagcatcagggcccgcccacaacgcccccgcctcccttcccgacggcctatccgccggagctgcagagcatcgtggtgcagccggaggtgacggtggagacgacgcactcggacagcaacaccaccaaggtgacggccacggccaacatcaaggtggagctggccatgcccggcagggcggtgcgcagctataactttacgagttag